(19) United States
(12) Patent Application Publication
(10) Pub. No.: US 2015/0073158 A1
(43) Pub. Date: Mar. 12, 2015

(54) METHOD FOR PREPARING FUROFURAN LIGNAN COMPOUND

(71) Applicants: Si Young Cho, Yongin-si (KR); Hyun Woo Jeong, Yongin-si (KR); Dae Bang Seo, Yongin-si (KR); Sang Jun Lee, Yongin-si (KR)

(72) Inventors: Si Young Cho, Yongin-si (KR); Hyun Woo Jeong, Yongin-si (KR); Dae Bang Seo, Yongin-si (KR); Sang Jun Lee, Yongin-si (KR)

(73) Assignee: AMOREPACIFIC CORPORATION, Seoul (KR)

(21) Appl. No.: 14/390,702

(22) PCT Filed: Apr. 9, 2013
(86) PCT No.: PCT/KR2013/002969

§ 371 Date: Oct. 3, 2014

(30) Foreign Application Priority Data
Apr. 10, 2012 (KR) 10-2012-0037273

(51) Int. Cl. C07D 493/04

(52) U.S. Cl. CPC C07D 493/04
USPC 549/464

(57) ABSTRACT

The present invention relates to a method for preparing a furofuran lignan compound, comprising a step of selecting and alkylating an epoxy alcohol compound and an optical isomer thereof. (+)-furofuran lignan and (−)-furofuran lignan, as well as an optical isomer thereof, can be selectively prepared by means of the method.

### TECHNICAL FIELD

The present disclosure relates to a novel method for preparing a furofuran lignan compound.

### BACKGROUND ART

Furofuran lignan, a naturally occurring lignan, is known to have an anticancer, antihypertensive or antioxidative effect, and thus many studies have been conducted to prepare such lignan with ease so that it may be used more widely. According to the related art, studies about methods for preparing furofuran lignan racemate have been conducted largely. However, methods for preparing each of (+)-furofuran lignan and an optical isomer thereof, i.e., (−)-furofuran lignan selectively have not been well known.

### DISCLOSURE

### Technical Problem

A technical problem to be solved by the present disclosure is to provide a method for preparing a furofuran lignan compound and an optical isomer thereof by using any one compound selected from epoxy alcohol compounds and optical isomers thereof.

### Technical Solution

In one general aspect, there is provided a method for preparing a furofuran lignan compound, including a step of O-alkylating any one compound selected from a compound represented by the following Chemical Formula 1 and an optical isomer thereof:

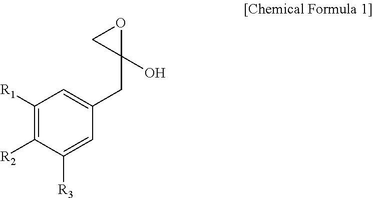

wherein each of R₁, R₂ and R₃ independently represents H, a C₁-C₃ alkoxy or methylenedioxy group together with the adjacent group.

### Advantageous Effects

The method for preparing a furofuran lignan compound according to the present disclosure allows production of a furofuran lignan compound as an individual optical isomer not a racemic mixture. Therefore, when a specific optical isomer of furofuran lignan compound is to be obtained, it can be produced in a simple manner without an additional separation process.

### BEST MODE

In an aspect of the present disclosure, there is provided a method for preparing a furofuran lignan compound, including a step of O-alkylating any one compound selected from a compound represented by the above Chemical Formula 1 and an optical isomer thereof. The method selects and uses any one compound selected from an epoxy alcohol compound such as a compound represented by Chemical Formula 1 and an optical isomer thereof, and thus can produce a furofuran lignan compound as an individual optical isomer not a racemate. In other words, the method according to the present disclosure allows asymmetric synthesis of a furofuran lignan compound.

According to an embodiment, the optical isomer of a compound represented by Chemical Formula 1 includes a compound represented by the following Chemical Formula 2 or 3. Particularly, the optical isomer of a compound represented by Chemical Formula 1 includes a compound represented by the following Chemical Formula 4 or 5:

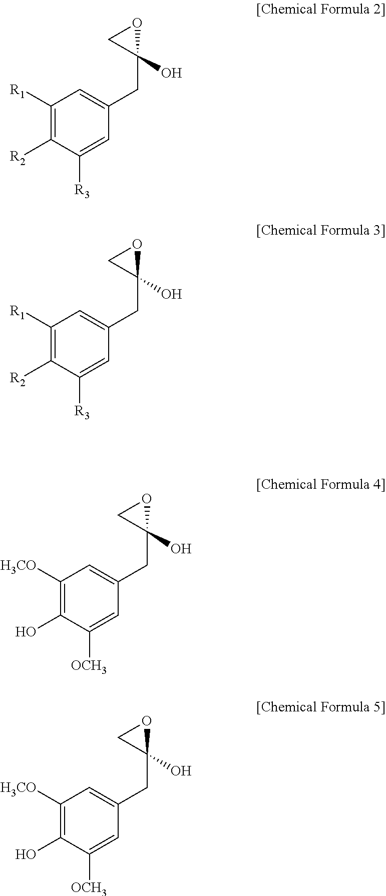

wherein each of R₁, R₂ and R₃ independently represents H, a C₁-C₃ alkoxy or methylenedioxy group together with the adjacent group.

According to another embodiment, the furofuran lignan compound may be represented by the following Chemical Formula 6:

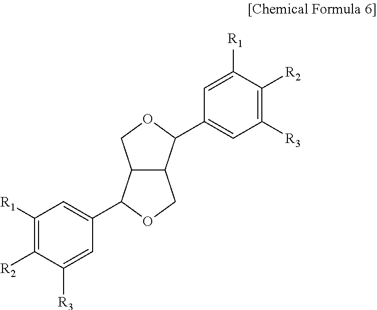

According to still another embodiment, the furofuran lignan compound includes syringaresinol, eudesmin, yangambin or sesamin, but is not limited thereto. The furofuran lignan compound obtained according to an aspect of the present disclosure includes a furofuran lignan compound or an isomer thereof. Herein, the isomer includes an optical isomer, particularly an isomer represented by the following Chemical Formula 7 or Chemical Formula 8, and more particularly, (+)-syringaresinol, (−)-syringaresinol, (+)-eudesmin, (−)-eudesmin, (+)-yangambin (−)-yangambin, (+)-sesamin or (−)-sesamin. Even more particularly, the furofuran lignan compound obtained by the method according to an aspect of the present disclosure includes (8S,8′S)-(−)-syringaresinol represented by the following Chemical Formula 9 or (8R,8′R)-(+)-syringaresinol by the following Chemical Formula 10:

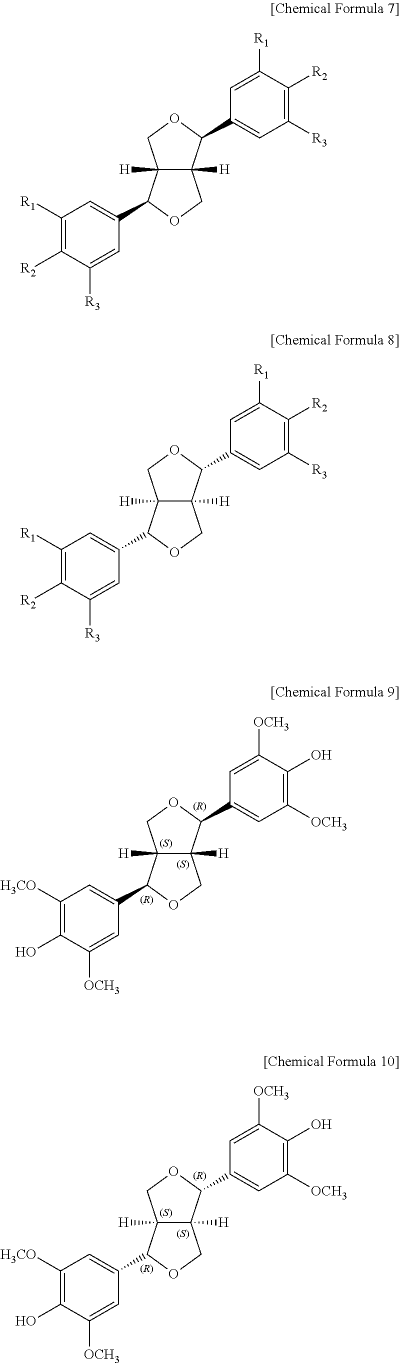

wherein each of R₁, R₂ and R₃ independently represents H, a C₁-C₃ alkoxy or methylenedioxy group together with the adjacent group.

In another aspect of the present disclosure, there is provided a method for preparing a furofuran lignan compound. In the method, the optical isomer of the compound represented by Chemical Formula 1 may be obtained by cyclization of a compound represented by the following Chemical Formula 11. Particularly, the optical isomer of the compound represented by Chemical Formula 1 may be obtained by reacting the compound represented by Chemical Formula 11 with (+)-diisopropyltryptamine (DIPT) or (−)-DIPT. According to an embodiment, the compound represented by Chemical Formula 11 is reacted with (+)-DIPT and (−)-DIPT to provide a compound represented by Chemical Formula 2 and Chemical Formula 3, respectively.

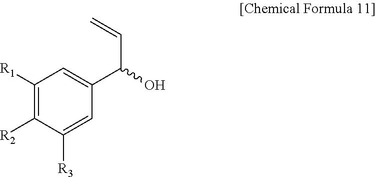

wherein each of R₁, R₂ and R₃ independently represents H, a C₁-C₃ alkoxy or methylenedioxy group together with the adjacent group.

According to an embodiment, the compound represented by Chemical Formula 11 is an allyl alcohol racemate that may be obtained by reacting benzaldehyde substituted with R₁, R₂ and R₃ with a vinyl compound.

In the method for preparing a furofuran lignan compound according to an aspect of the present disclosure, the step of O-alkylating any one compound selected from the compound represented by Chemical Formula 1 and an isomer thereof may be carried out by reacting any one compound selected from the compound represented by Chemical Formula 1 and an isomer thereof with a compound represented by the following Chemical Formula 12 to provide a compound represented by the following Chemical Formula 13 or an isomer thereof (Chemical Formula 14 or 15). According to another embodiment, when reacting a compound represented by Chemical Formula 2 or 3 among the isomers of the compound represented by Chemical Formula 1 with a compound represented by Chemical Formula 12, it is possible to provide a compound represented by Chemical Formula 14 or 15, respectively.

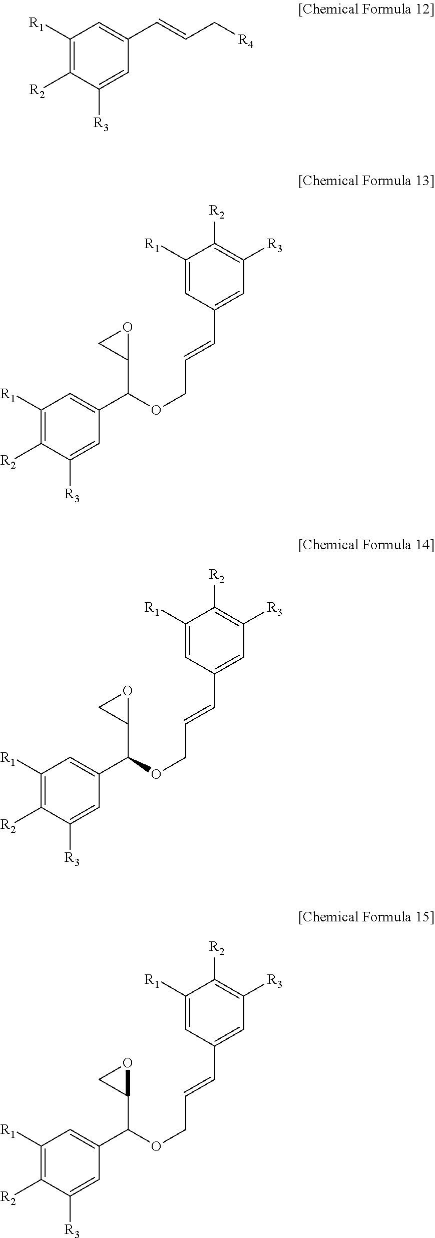

wherein each of R₁, R₂ and R₃ independently represents H, a C₁-C₃ alkoxy or methylenedioxy group together with the adjacent group, and R₄ represents a halogen group such as fluorine, bromine, chlorine or iodine.

According to another embodiment, the compound represented by Chemical Formula 12 may be obtained by converting the aldehyde group of benzaldehyde substituted with R₁, R₂ and R₃ into an unsaturated ester group, reducing the ester group into an unsaturated alcohol group, and substituting the —OH group of alcohol with a halogen group.

According to still another embodiment, the method for preparing a furofuran lignan compound may further include, after the step of preparing a compound represented by Chemical Formula 13 or an optical isomer thereof, a step of cyclizing the resultant compound represented by Chemical Formula 13 or an optical isomer thereof to provide a compound represented by the following Chemical Formula 16 or an optical isomer (Chemical Formula 17 or 18) thereof.

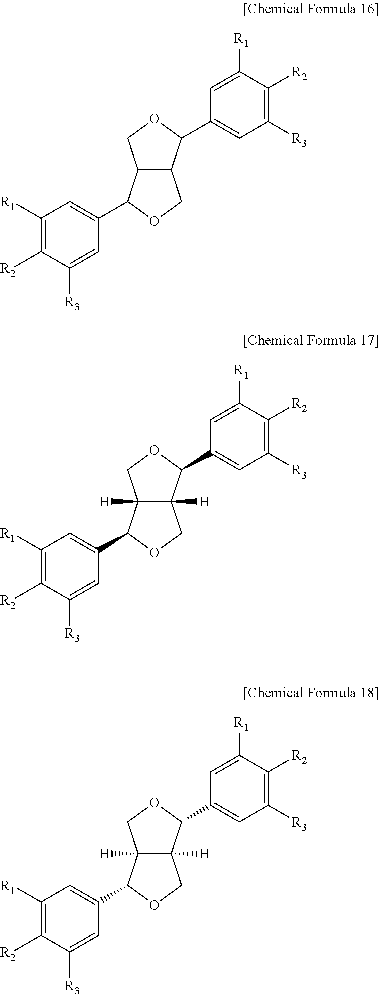

wherein each of R₁, R₂ and R₃ independently represents H, a C₁-C₃ alkoxy or methylenedioxy group together with the adjacent group.

According to still another embodiment, the above steps may be carried out by using the compounds represented by the above Chemical Formulae in which at least one of R₁, R₂ and R₃ is subjected to benzyl protection with halogenated benzene. In this case, after providing a compound represented by Chemical Formula 16, 17 or 18, the benzyl group is removed by treatment using Raney nickel, thereby producing a final furofuran lignan compound.

Hereinafter, the method for preparing a furofuran lignan compound is exemplified particularly by a method for preparing (−)-syringaresinol. First, a starting material, syringaldehyde (19), is subjected to benzyl protection to provide Compound 20, which in turn is reacted with vinyl magnesium bromide to provide racemic mixture (21) of secondary allyl alcohols. Next, Compound 21 is subjected to Sharpless kinetic resolution together with cumene hydroperoxide in the presence of titanium isopropoxide and (+)-DIPT at −20° C. to obtain Compound 22 and optically active epoxy alcohol compound (23). The absolute configuration of Compound 23 can be determined from the published reports and the absolute configuration of (−)-syringaresinol obtained from Compound 23. The resultant epoxy alcohol compound (23) is reacted with cinnamyl bromide (24) in THF-DMSO (10:1) in the presence of NaH to obtain O-alkylated compound (25). Then, Compound 25 is subjected to radical cyclization in the presence of bis(cyclopentadienyl)titanium (III) chloride (Cp₂TiCl), followed by treatment with iodine, to obtain a furofuran lignan compound (26). Herein, bis(cyclopentadienyl)titanium (III) chloride (Cp₂TiCl) can be obtained with ease by treating Cp₂TiCl₂ with activated zinc dust. Finally, the benzyl group of furofuran lignan compound (26) is removed by treatment with Raney nickel to obtain a final product, (−)-syringaresinol (Chemical Formula 9). See the following Reaction Scheme 1.

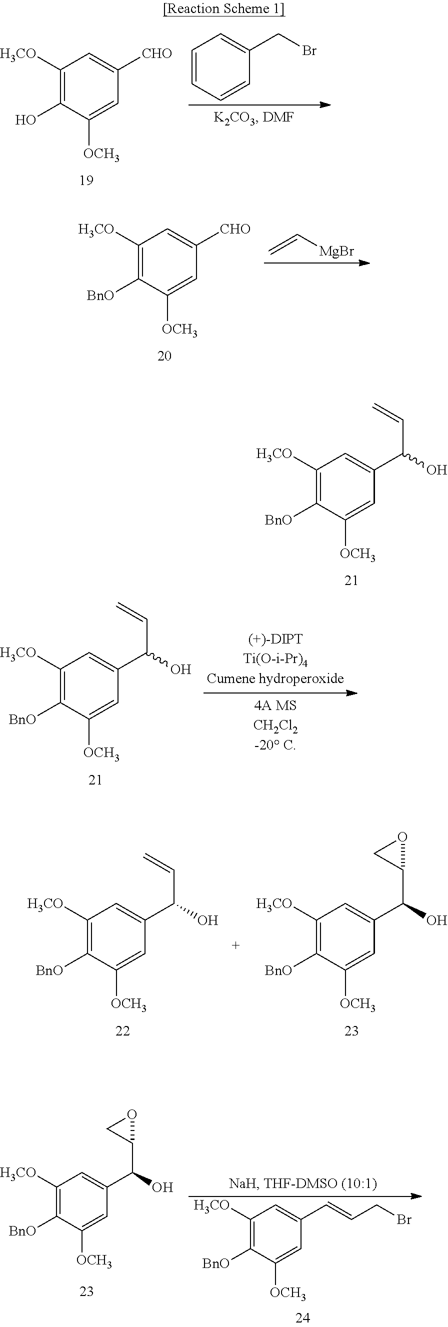

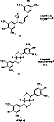

The method for preparing a furofuran lignan compound is also exemplified by a method for preparing (+)-syringaresinol. First, substantially in the same manner as the method for preparing (−)-syringaresinol, racemic mixture (21) of secondary allyl alcohols is subjected to Sharpless kinetic resolution together with cumene hydroperoxide in the presence of titanium isopropoxide and (−)-DIPT at −20° C. to obtain epoxy alcohol compound (28) having the opposite stereoconfiguration to epoxy alcohol compound (23). Then, epoxy alcohol compound (28) is used to carry out the remaining procedure substantially in the same manner as the method for preparing (−)-syringaresinol. In this manner, it is possible to obtain (+)-syringaresinol. See the following Reaction Scheme 2.

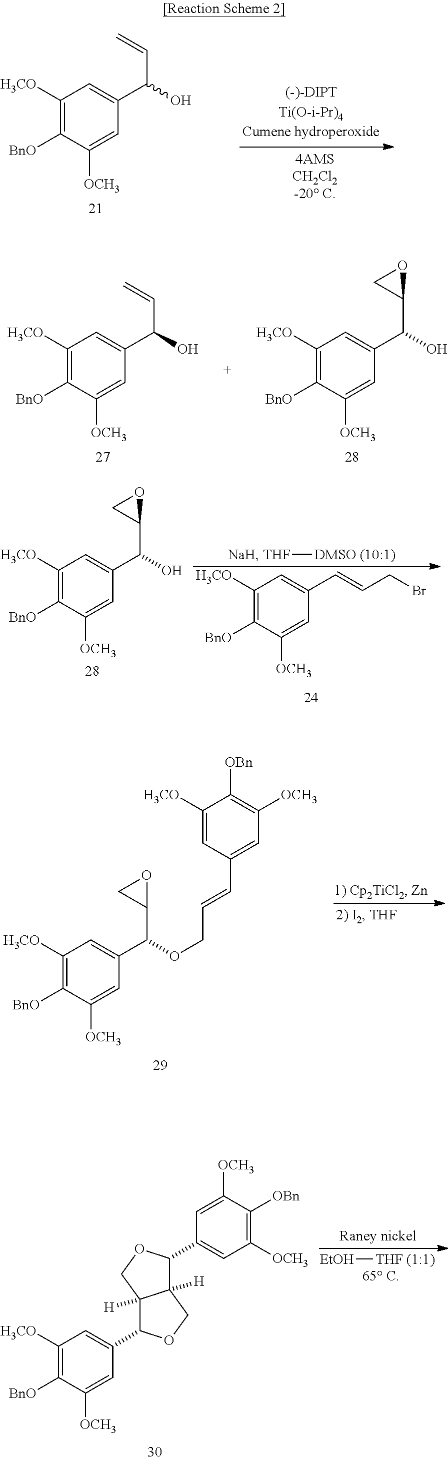

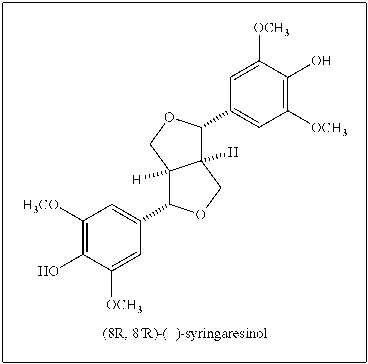

Meanwhile, cynnamyl bromide (24) may be obtained by converting Compound 20 into Compound (31) having an unsaturated ester group through the Horner-Wadsworth-Emmons olefination, reducing Compound (31) with DIBAL-H to provide Compound (32) having an unsaturated alcohol group, and then substituting OH with Br by using PBr₃. See the following Reaction Scheme 3.

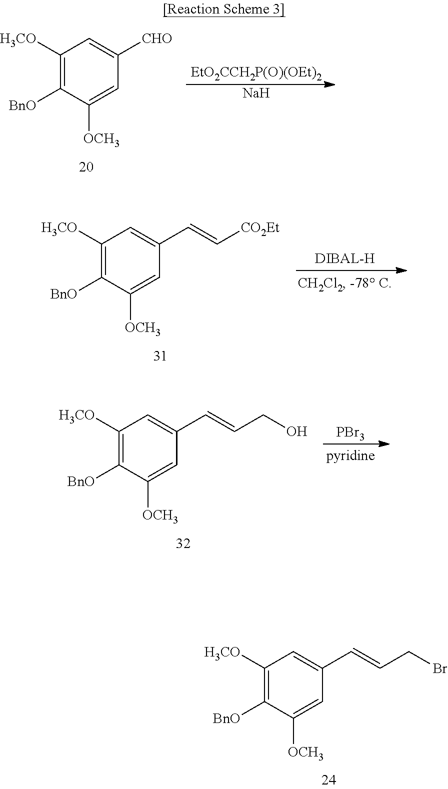

The examples now will be described more fully hereinafter. The following examples are for illustrative purposes only and not intended to limit the scope of the present disclosure.

### Example 1

### Preparation of (−)-Syringaresinol (Chemical Formula 9)

### (1) Preparation of 4-(benzyloxy)-3,5-dimethoxybenzaldehyde (Compound 20)

Under nitrogen atmosphere, syringaldehyde (Compound 19) (3 g, 16.47 mmol) and potassium carbonate (6.83 g, 49.4 mmol) are dissolved into DMF (16 mL), benzyl bromide (2.94 mL, 24.7 mmol) is introduced thereto, and the reaction mixture is agitated overnight at room temperature. After determining the completion of reaction by TLC, the reaction solution is extracted with water and ethyl acetate. The extracted organic layer is washed with water three times, dried with anhydrous MgSO₄, and filtered through a filter. The filtrate is concentrated and purified by column chromatography (5:1 hexane/EtOAc) to obtain Compound 20 as a white solid (4.45 g, 99.2%). NMR data of Compound 20 is shown below.

¹H NMR (300 MHz, CDCl₃) δ 3.90 (s, 6H), 5.13 (s, 2H), 7.11 (s, 2H), 7.29-7.48 (m, 5H), 9.86 (s, 1H); ¹³C NMR (75 MHz, CDCl₃) δ 56.2, 75.0, 106.7, 128.1, 128.2, 128.4, 128.6, 131.9, 137.2, 191.1.

### (2) Preparation of 1-(4-benzyloxy)-3,5-dimethoxyphenyl)prop-2-en-1-ol (Compound 21)

Under nitrogen atmosphere, the resultant Compound 20 (4.04 g, 14.8 mmol) is dissolved into anhydrous THF (30 mL) and the solution is cooled to −78° C. Next, vinyl magnesium bromide (1M THF solution, 17.8 mL) is added thereto, and the reaction mixture is agitated at −78° C. for 10 minutes, warmed to 0° C. and further agitated for 2 hours. After determining the completion of the reaction by TLC, the reaction solution is quenched with saturated aqueous NH₄Cl solution, and extracted with ethyl acetate. The extracted organic layer is washed with water three times, dried with anhydrous MgSO₄ and filtered through a filter. The filtrate is concentrated and purified by column chromatography (4:1 hexane/EtOAc) to obtain Compound 21 as a light yellow solid (3.97 g, 89.1%). NMR data of Compound 21 is shown below.

¹H NMR (300 MHz, CDCl₃) δ 3.83 (s, 6H), 4.99 (s, 2H), 5.14 (m, 1H), 5.22 (ddd, J=10.5, 1.2, 1.2 Hz, 1H), 5.37 (ddd, J=16.8, 1.8, 1.8 Hz, 1H), 6.05 (ddd, J=16.5, 10.5, 6.0 Hz, 1H), 6.60 (s, 2H), 7.28-7.51 (m, 5H); ¹³C NMR (75 MHz, CDCl₃) δ 56.1, 75.0, 75.4, 102.5, 103.4, 115.3, 127.8, 128.1, 128.4, 137.9, 138.3, 140.0, 153.6.

### (3) Preparation of (S)-(4-(benzyloxy)-3,5-dimethoxyphenyl) ((S)-oxiran-2-yl)methanol) (Compound 23)

Under nitrogen atmosphere, anhydrous methylene chloride (45 mL) is introduced to dried powdered 4 Å molecular sieve (10.5 g) and the mixture is cooled to −20° C. Next, (+)-DIPT (diisopropyltryptamine) (1.18 mL, 5.99 mmol) is added thereto and titanium isopropoxide (1.48 mL, 4.99 mmol) is further added gradually thereto, followed by agitation for 30 minutes. After 30 minutes, cumene hydroxide (1.85 mL, 80%, 5.99 mmol) is added thereto, followed by agitation for 30 minutes. Then, Compound 21 (3 g, 9.99 mmol) dissolved in anhydrous methylene chloride (10 mL) is added dropwise thereto. After the resultant mixture is agitated at −20° C. for 5 hours, the mixture is warmed to 0° C. and agitated overnight. After determining the completion of the reaction by TLC, 10% NaOH solution (30 mL, in saturated NaCl solution) is added and the reaction mixture is agitated for 3 hours, followed by filtration. The filtrate is extracted with methylene chloride and washed with brine. The resultant product is dried with anhydrous Na₂SO₄ and filtered through a filter, and the filtrate is concentrated and purified by column chromatography (3:1 hexane/EtOAc) to obtain Compound 22 (1.65 g, 55.0%, red oil) and Compound 23 (1.06 g, 33.5%, red oil). NMR data of Compound 23 is shown below.

[α]_{D}+52.9° (c 1.0, CHCl₃); ¹H NMR (300 MHz, CDCl₃) δ 2.20 (s, 1H), 2.79 (dd, J=5.1, 3.9 Hz, 1H), 2.95 (dd, J=5.1, 2.7 Hz, 1H), 3.23 (q, J=3.9 Hz, 1H), 3.84 (s, 6H), 4.85 (d, J=3.3 Hz, 1H), 5.00 (s, 2H), 6.62 (s, 2H), 7.29-7.51 (m, 5H); ¹³C NMR (75 MHz, CDCl₃) δ 43.7, 55.0, 56.2, 71.1, 75.0, 103.4, 127.8, 128.1, 128.4, 135.2, 137.8, 153.7.

### (4) Preparation of 2-(4-(S)-(((E)-3-(4-(benzyloxy)-3,5-dimethoxyphenyl) allyl)oxy) ((S)-oxiran-2-yl)methyl)-2,6-dimethoxyphenoxy)tetrahydro-2H-pyran (Compound 25)

Under nitrogen atmosphere, dried THF/DMSO (10:1) (20 mL) is added to NaH (0.633 g, 60% dispersion, 15.82 mmol) and the mixture is cooled to 0° C. Next, Compound 23 (0.5 g, 1.58 mmol) dissolved in THF (10 mL) is added thereto, followed by agitation. Then, cinnamyl bromide (Compound 24) (1.15 g, 3.16 mmol) dissolved in THF (10 mL) is added thereto, and then the resultant mixture is agitated at 0° C. for 30 minutes, warmed to room temperature, and agitated overnight. After determining the completion of the reaction by TLC, the reaction solution is quenched with water and extracted with ethyl acetate. The extracted organic layer is washed with water three times, dried with anhydrous MgSO₄ and filtered through a filter. The filtrate is concentrated and purified by column chromatography (3:1 hexane/EtOAc) to obtain Compound 25 (760 mg, 80.27%) as light yellow oil. NMR data of Compound 25 is shown below.

¹H NMR (300 MHz, CDCl₃) δ 2.82 (m, 2H), 3.20 (dt, J=3.3, 3.9 Hz, 1H), 3.83 (s, 6H), 3.85 (s, 6H), 4.08 (m, 2H), 4.33 (d, J=4.5 Hz, 1H), 5.02 (s, 2H), 5.03 (s, 2H), 6.19 (dt, J=15.9, 6.0 Hz, 1H), 6.48 (d, J=16.2 Hz, 1H), 6.59 (s, 2H), 6.61 (s, 2H), 7.27-7.53 (m, 10H); ¹³C NMR (75 MHz, CDCl₃) δ 45.2, 54.4, 56.1, 56.1, 69.6, 74.9, 75.0, 80.1, 103.7, 104.3, 125.1, 127.7, 127.8, 128.1, 128.3, 128.4, 132.3, 132.7, 134.0, 136.9, 137.7, 137.8, 153.6, 153.7.

### (5) Preparation of Compound 26

Under nitrogen atmosphere, Cp₂TiCl₂ (0.182 g, 1.463 mmol) dissolved in THF (20 mL) is introduced to a flask containing activated zinc dust (0.146 g, 4.44 mmol) and the resultant mixture is agitated for 1 hour to provide Cp₂TiCl (green solution). In another flask, Compound 25 (0.19 g, 0.635 mmol) is dissolved in THF (16 mL), the temperature is adjusted to 60° C., and then the preliminarily formed Cp₂TiCl solution is added thereto for 20 minutes by using a cannula. After carrying out agitation for 20 minutes, I₂ (0.105 g, 0.825 mmol) dissolved in THF (4 mL) is added and the reaction solution is agitated for 1 hour. After determining the completion of the reaction by TLC, saturated aqueous NH₄Cl solution is added to quench the reaction, followed by extraction with diethyl ether. The extracted organic layer is washed with 10% Na₂S₂O₃ solution and brine solution three times, dried with anhydrous Na₂SO₄, and filtered through a filter. The filtrate is concentrated and purified by column chromatography (3:1 hexane/EtOAc) to obtain Compound 26 (62 mg, 32.6%) as light red oil. NMR data of Compound 26 is shown below.

¹H NMR (300 MHz, CDCl₃) δ 3.11 (m, 2H), 3.84 (s 12H), 3.94 (dd, J=9.3, 3.6 Hz, 2H), 4.31 (dd, J=8.7, 6.6 Hz, 2H), 4.75 (d, J=4.2 Hz, 2H), 4.99 (s, 4H), 6.57 (s, 4H), 7.29-7.51 (m, 10H); ¹³C NMR (75 MHz, CDCl₃) δ 54.3, 56.2, 72.0, 75.0, 86.0, 103.0, 127.8, 128.1, 128.4, 136.8, 137.8, 153.7.

### (6) Preparation of (−)-Syringaresinol (Chemical Formula 9)

Compound 26 (90 mg, 0.152 mmol) is dissolved into ethanol/THF (1:1) (9 mL) and the temperature is adjusted to 65° C. Next, Raney nickel (0.15 g) is introduced thereto and the resultant mixture is agitated at 65° C. for 1 hour. After determining the completion of the reaction by TLC, the reaction solution is filtered by using acetone and celite, and then the filtrate is concentrated and purified by column chromatography (1:1 hexane/EtOAc) to obtain a light yellow sold (50 mg, 78.7%). It can be seen from the following NMR data and an [α]_{D} value of −38.5° (c 0.1, CHCl₃) that the resultant compound is (8S,8′S-(−)-syringaresinol).

¹H NMR (400 MHz, CDCl₃) δ 3.09 (m, 2H), 3.90 (s 12H), 3.91 (m, 2H), 4.28 (dd, J=8.8, 6.8 Hz, 2H), 4.73 (d, J=4.4 Hz, 2H), 5.51 (s, 2H), 6.58 (s, 4H); ¹³C NMR (100 MHz, CDCl₃) δ 54.3, 56.4, 71.8, 86.1, 102.7, 132.1, 134.3, 147.1.

### Example 2

### Preparation of (+)-Syringaresinol (Chemical Formula 10)

### (1) Preparation of (R)-(4-(benzyloxy)-3,5-dimethoxyphenyl) ((R)-oxiran-2-yl)methanol (Compound 28)

Compound 28 is obtained from Compound 21 substantially in the same manner as the method for preparing Compound 23 in Example 1, except that (−)-DIPT is used instead of (+)-DIPT. NMR data of Compound 28 is shown below.

[α]_{D} −47.5° (c 1.0, CHCl₃); ¹H NMR (300 MHz, CDCl₃) δ 2.32 (d, J=2.1 Hz, 1H), 2.79 (dd, J=4.8, 3.9 Hz, 1H), 2.94 (dd, J=4.8, 2.7 Hz, 1H), 3.22 (q, J=3.3 Hz, 1H), 3.84 (s, 6H), 4.82 (t, J=2.1 Hz, 1H), 5.00 (s, 2H), 6.61 (s, 2H), 7.29-7.51 (m, 5H); ¹³C NMR (75 MHz, CDCl₃) δ 43.73, 54.98, 56.14, 71.11, 74.99, 76.57, 77.00, 77.42, 103.39, 127.78, 128.11, 128.41, 135.20, 136.84, 137.78, 153.69.

### (2) Preparation of (2-(4-((R)-(((E)-3-(4-(benzyloxy)-3,5-dimethoxyphenyl)allyl)oxy) ((R)-oxiran-2-yl)methyl)-2,6-dimethoxyphenoxy)tetrahydro-2H-pyran (Compound 29)

Compound 29 is obtained by reacting Compound 24 with Compound 28 substantially in the same manner as the method for preparing Compound 25 in Example 1. NMR data of Compound 29 is shown below.

¹H NMR (300 MHz, CDCl₃) δ 2.82 (m, 2H), 3.19 (dt, J=2.7, 3.9 Hz, 1H), 3.83 (s, 6H), 3.84 (s, 6H), 4.12 (m, 2H), 4.32 (d, J=5.1 Hz, 1H), 5.01 (s, 2H), 5.02 (s, 2H), 6.18 (dt, J=15.9, 6.0 Hz, 1H), 6.47 (d, J=17.1 Hz, 1H) 6.59 (d, J=4.8 Hz, 4H), 7.28-7.52 (m, 10H); ¹³C NMR (75 MHz, CDCl₃) δ 45.3, 54.4, 56.1, 56.2, 69.7, 75.0, 75.1, 80.1, 103.7, 104.4, 125.1, 127.8, 128.1, 128.4, 128.5, 132.3, 132.8, 134.0, 137.9, 153.6, 156.7.

(3) Preparation of Compound 30 Compound 30 is obtained from Compound 29 substantially in the same manner as the method for preparing Compound 26 in Example 1. NMR data of Compound 30 is shown below.

¹H NMR (300 MHz, CDCl₃) δ 3.11 (m, 2H), 3.84 (s, 12H), 3.93 (dd, J=9.4, 3.6 Hz, 2H), 4.31 (dd, J=9.3, 7.2 Hz, 2H), 4.75 (d, J=3.9 Hz, 2H), 4.99 (s, 4H), 6.57 (s, 4H), 7.28-7.51 (m, 10H); ¹³C NMR (75 MHz, CDCl₃) δ 54.3, 56.2, 72.0, 75.0, 86.0, 103.0, 127.8, 128.1, 128.4, 136.8, 137.8, 153.7.

### (4) Preparation of (+)-Syringaresinol (Chemical Formula 10)

(+)-Syringaresinol is obtained from Compound 30 substantially in the same manner as the method for preparing (−)-syringaresinol (Chemical Formula 9) in Example 1. It can be seen from the following NMR data and an [α]_{D} value of +40.9° (c 0.1, CHCl₃) that the resultant compound is (8R,8′R-(+)-syringaresinol).

[α]_{D} +40.9° (c 0.1, CHCl₃); ¹H NMR (400 MHz, CDCl₃) δ 3.10 (m, 2H), 3.90 (s, 12H), 3.90 (m, 2H), 4.28 (dd, J=8.8, 6.8 Hz, 2H), 4.73 (d, J=4.4 Hz, 2H), 5.48 (s, 2H), 6.59 (s, 2H); ¹³C NMR (100 MHz, CDCl₃) δ 54.4, 56.4, 71.8, 86.1, 102.7, 132.1, 134.3, 147.1.

### Preparation Example

### Preparation of Compound 24

Compound 24 used for preparing Compound 25 or Compound 29 in Example 1 and Example 2 is obtained by the following method.

### (1) Preparation of ((E)-ethyl 3-(4-(benzyloxy)-3,5-dimethoxyphenyl) acrylate (Compound 31)

To a 250 mL round-bottom flask, 25 mL of THF and 60% NaH (1.575 g, 39.387 mmol) are introduced and triethyl phosphonoacetate (7.064 g, 31.509 mmol) dissolved in 50 mL of THF is further introduced thereto, followed by agitation at room temperature for 30 minutes. After cooling the mixture to 0° C., Compound 20 (7.15 g, 26.258 mmol) dissolved in 25 mL of THF is added and reaction is carried out for 3 hours. The reaction mixture is diluted with water and EtOAc and extracted with EtOAc three times. The organic layer is washed with brine and dried with MgSO₄. After removing the solvent, the resultant product is purified by column chromatography (5:1 hexane/EtOAc) to obtain 4.12 g (12.033 mmol, 45.83%) of Compound 31 as a white solid. NMR data of Compound 31 is shown below.

¹H NMR (300 MHz, CDCl₃) δ 1.34 (t J=7.2 Hz, 3H), 3.85 (s, 6H), 4.27 (q, J=7.2 Hz, 2H), 5.05 (s, 2H), 6.34 (d, J=15.3 Hz, 1H), 6.74 (s, 2H), 7.29-7.49 (m, 5H), 7.60 (d, J=15.9 Hz, 1H); ¹³C NMR (75 MHz, CDCl₃) δ 14.3, 56.1, 60.5, 75.1, 105.1, 105.3, 117.5, 127.9, 128.2, 128.4, 130.1, 137.5, 144.6, 153.7, 166.9.

### (2) Preparation of ((E)-3-(4-benzyloxy)-3,5-dimethoxyphenyl)prop-2-en-1-ol (Compound 32)

To a 100 mL round-bottom flak, Compound 31 (2 g, 5.841 mmol) and dichloromethane (10 mL) are introduced and the mixture is cooled to −78° C. Next, 1.0M DIBAL-H toluene solution (14.603 mL, 14.603 mmol) is added dropwise thereto by using a syringe. After carrying out reaction for 30 minutes, the reaction mixture is warmed to 0° C. and reaction is further carried out for 1 hour. After determining the completion of the reaction by TLC, the reaction mixture is quenched with saturated NH₄Cl solution at room temperature, followed by agitation of 10 minutes. The reaction mixture is filtered with Et₂O and extracted with Et₂O three times. The organic layer is washed with brine and dried with MgSO₄. After removing the solvent, the resultant product is purified by column chromatography (2:1 hexane/EtOAc) to obtain 1.57 g (5.227 mmol, 89.49%) of Compound 32 as a white solid. NMR data of Compound 32 is shown below.

¹H NMR (300 MHz, CDCl₃) δ 3.83 (s, 6H), 4.32 (dd, J=5.7, 1.2 Hz, 2H), 5.01 (s, 2H), 6.29 (dt, J=16.2, 5.4 Hz, 1H), 6.54 (d, J=15.9 Hz, 1H), 6.61 (s, 2H), 7.28-7.50 (m, 5H); ¹³C NMR (75 MHz, CDCl₃) δ 56.10, 63.66, 75.08, 103.74, 105.13, 127.81, 127.94, 128.12, 128.47, 131.24, 132.47, 137.80, 153.62.

### (3) Preparation of (E)-2-benzyloxy)-5-(3-bromoprop-1-en-1-yl)-1,3-dimethoxybenzene (Compound 24)

Compound 32 (0.8 g, 2.664 mmol) is dissolved into Et₂O (25 mL) and the solution is cooled to 0° C. Next, pyridine (43 μl, 0.524 mmol) is added thereto and phosphorus tribromide (0.25 mL, 2.664 mmol) is added gradually thereto. After carrying out reaction for 1 hour, the reaction mixture is quenched with saturated NaHCO₃ solution. The reaction mixture is extracted with Et₂O three times, and the organic layer is washed with brine and dried with MgSO₄. The resultant white solid is used for preparing Compound 25 or Compound 29 without further purification. NMR data of Compound 24 is shown below.

¹H NMR (300 MHz, CDCl₃) δ 3.84 (s, 6H), 4.17 (dd, J=7.2, 1.8 Hz, 2H), 5.01 (s, 2H), 6.31 (dt, J=15.9, 7.8 Hz, 1H), 6.57 (d, J=15.3 Hz, 1H), 6.60 (s, 2H), 7.28-7.49 (m, 5H); ¹³C NMR (75 MHz, CDCl₃) δ 33.45, 56.14, 75.09, 104.03, 124.58, 127.86, 128.14, 128.49, 134.64, 153.66.

1. A method for preparing a furofuran lignan compound, comprising a step of O-alkylating any one compound selected from a compound represented by the following Chemical Formula 1 and an optical isomer thereof: 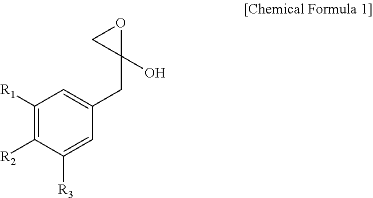
wherein each of R₁, R₂ and R₃ independently represents H, a C₁-C₃ alkoxy or methylenedioxy group together with the adjacent group.

2. The method for preparing a furofuran lignan compound according to claim 1, wherein the furofuran lignan compound comprises a compound represented by the following Chemical Formula 7 or 8: 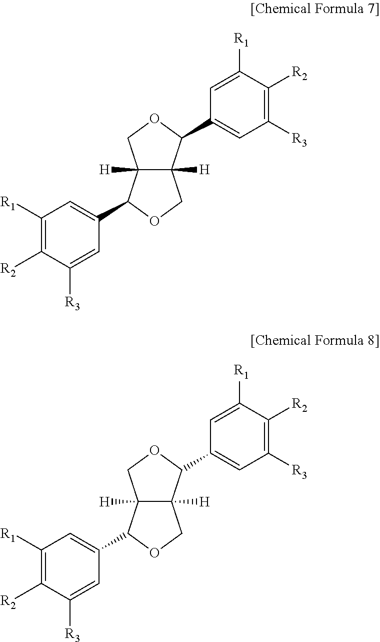
wherein each of R₁, R₂ and R₃ independently represents H, a C₁-C₃ alkoxy or methylenedioxy group together with the adjacent group.

3. The method for preparing a furofuran lignan compound according to claim 1, wherein the optical isomer of a compound represented by Chemical Formula 1 comprises a compound represented by the following Chemical Formula 2 or 3: 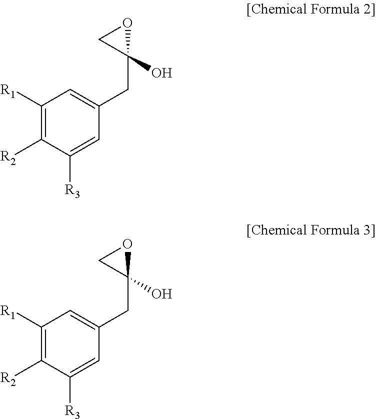
wherein each of R₁, R₂ and R₃ independently represents H, a C₁-C₃ alkoxy or methylenedioxy group together with the adjacent group.

4. The method for preparing a furofuran lignan compound according to claim 1, wherein the optical isomer of the compound represented by Chemical Formula 1 is obtained by reacting a compound represented by the following Chemical Formula 11 with (+)-diisopropyltryptamine (DIPT) or (−)-DIPT: 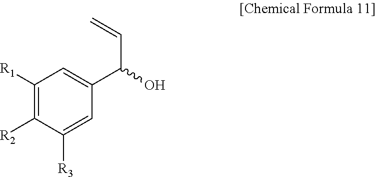
wherein each of R₁, R₂ and R₃ independently represents H, a C₁-C₃ alkoxy or methylenedioxy group together with the adjacent group.

5. The method for preparing a furofuran lignan compound according to claim 1, wherein the step of O-alkylating any one compound selected from the compound represented by Chemical Formula 1 and an isomer thereof is carried out by reacting any one compound selected from the compound represented by Chemical Formula 1 and an isomer thereof with a compound represented by the following Chemical Formula 12 to provide a compound represented by the following Chemical Formula 13 or an isomer thereof: 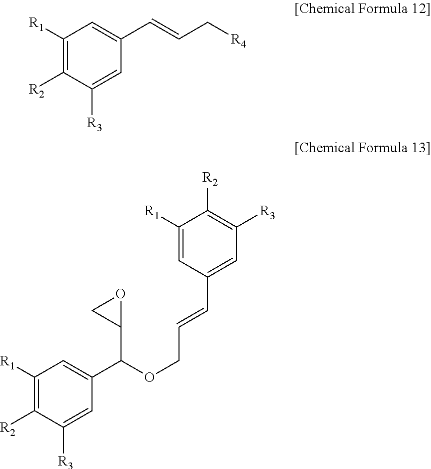
wherein each of R₁, R₂ and R₃ independently represents H, a C₁-C₃ alkoxy or methylenedioxy group together with the adjacent group, and R4 represents a halogen group such as fluorine, bromine, chlorine or iodine.

6. The method for preparing a furofuran lignan compound according to claim 5, which further comprises a step of cyclizing a compound represented by Chemical Formula 13 or an optical isomer thereof to provide a compound represented by the following Chemical Formula 16 or an optical isomer thereof: 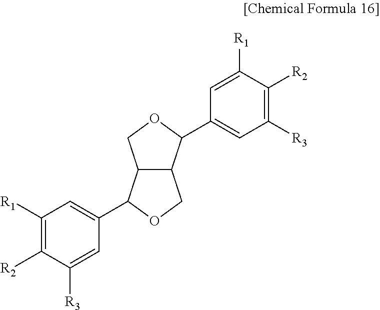
wherein each of R₁, R₂ and R₃ independently represents H, a C₁-C₃ alkoxy or methylenedioxy group together with the adjacent group.

7. The method for preparing a furofuran lignan compound according to claim 1, wherein at least one compound selected from Chemical Formula 1, Chemical Formula 2, Chemical Formula 3, Chemical Formula 7, Chemical Formula 8, Chemical Formula 11, Chemical Formula 12, Chemical Formula 13 and Chemical Formula 16 comprises a compound in which at least one of R₁, R₂ and R₃ is protected with a benzyl group.

8. The method for preparing a furofuran lignan compound according to claim 2, wherein at least one compound selected from Chemical Formula 1, Chemical Formula 2, Chemical Formula 3, Chemical Formula 7, Chemical Formula 8, Chemical Formula 11, Chemical Formula 12, Chemical Formula 13 and Chemical Formula 16 comprises a compound in which at least one of R₁, R₂ and R₃ is protected with a benzyl group.

9. The method for preparing a furofuran lignan compound according to claim 3, wherein at least one compound selected from Chemical Formula 1, Chemical Formula 2, Chemical Formula 3, Chemical Formula 7, Chemical Formula 8, Chemical Formula 11, Chemical Formula 12, Chemical Formula 13 and Chemical Formula 16 comprises a compound in which at least one of R₁, R₂ and R₃ is protected with a benzyl group.

10. The method for preparing a furofuran lignan compound according to claim 4, wherein at least one compound selected from Chemical Formula 1, Chemical Formula 2, Chemical Formula 3, Chemical Formula 7, Chemical Formula 8, Chemical Formula 11, Chemical Formula 12, Chemical Formula 13 and Chemical Formula 16 comprises a compound in which at least one of R₁, R₂ and R₃ is protected with a benzyl group.

11. The method for preparing a furofuran lignan compound according to claim 5, wherein at least one compound selected from Chemical Formula 1, Chemical Formula 2, Chemical Formula 3, Chemical Formula 7, Chemical Formula 8, Chemical Formula 11, Chemical Formula 12, Chemical Formula 13 and Chemical Formula 16 comprises a compound in which at least one of R₁, R₂ and R₃ is protected with a benzyl group.

12. The method for preparing a furofuran lignan compound according to claim 6, wherein at least one compound selected from Chemical Formula 1, Chemical Formula 2, Chemical Formula 3, Chemical Formula 7, Chemical Formula 8, Chemical Formula 11, Chemical Formula 12, Chemical Formula 13 and Chemical Formula 16 comprises a compound in which at least one of R₁, R₂ and R₃ is protected with a benzyl group.
